(19) United States
(12) Patent Application Publication
(10) Pub. No.: US 2024/0342135 A1
(43) Pub. Date: Oct. 17, 2024

(54) USE OF NITRATE AND VITAMIN C MICROCAPSULE FOR TREATING SICCA SYNDROME

(71) Applicants: CAPITAL MEDICAL UNIVERSITY, Beijing (CN); BEIJING STOMATOLOGICAL HOSPITAL, CAPITAL MEDICAL UNIVERSITY, Beijing (CN)

(72) Inventors: Songling WANG, Beijing (CN); Yuji WANG, Beijing (CN); Lizheng QIN, Beijing (CN); Liang HU, Beijing (CN); Lei HU, Beijing (CN); Guoqing LI, Beijing (CN)

(73) Assignees: CAPITAL MEDICAL UNIVERSITY, Beijing (CN); BEIJING STOMATOLOGICAL HOSPITAL, CAPITAL MEDICAL UNIVERSITY, Beijing (CN)

(21) Appl. No.: 18/685,073

(22) PCT Filed: Mar. 25, 2022
(86) PCT No.: PCT/CN2022/083009

§ 371 Date: Feb. 20, 2024

(30) Foreign Application Priority Data
Aug. 19, 2021 (CN) 202110951708.5

(51) Int. Cl. A61K 31/375; A61K 9/50; A61K 33/00; A61P 1/00

(52) U.S. Cl. CPC A61K 31/375; A61K 9/5036; A61K 9/5042; A61K 33/00; A61P 1/00

(57) ABSTRACT

Disclosed is the use of a nitrate and vitamin C microcapsule in the preparation of a drug for treating sicca syndrome. The microcapsule comprises a wall material and a core material encapsulated in the wall material, wherein the core material comprises vitamin C and nitrate, and a molar ratio of nitrate ions to vitamin C is 1:1 to 5:1.

### CROSS-REFERENCE TO RELATED APPLICATION

This application claims the benefit of Chinese Application No. 2021109517085 filed on Aug. 19, 2021. The application No. 2021109517085 is hereby incorporated by reference in its entirety.

### TECHNICAL FIELD

The invention belongs to the field of pharmaceutical preparation and medicine, and particularly relates to the use of a microcapsule of nitrate and vitamin C in treating sicca syndrome.

### DESCRIPTION OF RELATED ART

Sicca syndrome (SS) is a chronic inflammatory autoimmune disease mainly involving exocrine glands, also known as autoimmune exocrine gland epithelial inflammation or autoimmune exocrine disease. Because of the substantially damaged salivary glands and lacrimal glands, xerostomia and keratitis sicca are common in patients with SS. Because the saliva secretion is reduced, patients feel dry mouth, foreign body sensation and burning sensation. When chewing food, especially dry food, they can not form food balls and affect swallowing. Little saliva secretion leads to slight scouring effect on teeth and oral mucosa, deteriorating oral self-cleaning. Therefore, patients with xerostomia have a higher incidence of caries, and some even have rampant caries. The sense of taste in most xerostomia patients is also affected, which can not effectively stimulate appetite, and can affect the function of the whole digestive system.

At present, there is no effective treatment for SS in clinic, only symptomatic treatment can be adopted to alleviate dry mouth and reduce complications.

The combination of nitrate and vitamin C has been proven to have anti-tumor activity. However, there is no report on the use of the composition in treating SS so far. Vitamin C is very unstable. Environmental factors, such as temperature, pH, oxygen, metal ions, ultraviolet radiation and X-ray can affect the stability of vitamin C. In addition, vitamin C and nitrates have good water solubility, and their half-life in vivo is only 2 hours after oral administration, so it is difficult to maintain effective blood concentration. These are the problems that are urgently demanded to be solved in the further development of compositions comprising vitamin C and nitrates.

### SUMMARY

In order to overcome the defects of the prior art, the invention provides the use of a microcapsule of nitrate and vitamin C in treating SS.

Therefore, the invention adopts the following technical scheme:
the use of a microcapsule of nitrate and vitamin C in the preparation of a medicament for treating SS, wherein The microcapsule comprises a wall material and a core material encapsulated in the wall material, wherein the core material comprises vitamin C and nitrate, and the molar ratio of the nitrate ions to vitamin C is in the range from 1:1-5:1.

As a preferred embodiment, the invention provides the use of the microcapsule of the nitrate and the vitamin C in the preparation of a medicament for treating xerostomia caused by SS.

Preferably, the molar ratio of the nitrate ions to vitamin C is in the range from 1:1-4:1.

More preferably, the molar ratio of the nitrate ions to vitamin C is 4:1.

Preferably, the nitrate is selected from the group consisting of sodium nitrate and/or potassium nitrate, and more preferably is sodium nitrate.

Preferably, the wall material comprises pectin and sodium carboxymethyl cellulose.

Preferably, the core material further comprises chitosan.

Preferably, the chitosan is chitosan 3000.

As a preferred embodiment, the microcapsule comprises the following raw materials in parts by weight:
Pectin 0.7-1.2 parts by weight, sodium carboxymethyl cellulose 0.7-1.2 parts by weight, chitosan 1 part by weight, and nitrate and vitamin C (in total) 0.3-1.2 parts by weight;
Wherein the chitosan is chitosan 3000.

Preferably, the mass ratio of pectin, sodium carboxymethyl cellulose and chitosan is 0.85:0.85:1.

Preferably, the microcapsules are prepared by the following steps:
I. Preparing the raw materials based on their proportions;
II. Preparing the core material solution
   dissolving vitamin C, nitrate and chitosan in water to a final concentration of nitrate of about 4 mg/mL to obtain the core material solution;
III. Preparing the wall material solution
   uniformly mixing the wall material with water to a total mass percent concentration of the wall material in a range from 1.5 to 2.5% to obtain the wall material solution;
IV. Preparing the microcapsule
   uniformly mixing the core material solution prepared in step II and the wall material solution prepared in step III, freeze-drying, and crushing to obtain the microcapsules.

Preferably, the specific operation of the step II is as follows:
dissolving said parts by weight of vitamin C in water in a 20-25° C. water bath in dark, cooling to 2-4° C., adding said parts by weight of nitrate and chitosan, stirring to dissolve them to a final concentration of nitrate of about 4 mg/mL to obtain the core material solution.

Preferably, the specific operation of the step III is:
dissolving said parts by weight of sodium carboxymethyl cellulose in water at 70-80° C., and stirring or homogenizing under high pressure to obtain solution A; dissolving said parts by weight of pectin in water at 45-55° C. to obtain solution B, wherein the volume of the solution A is similar to that of the solution B; mixing the solution A and the solution B and stirring to homogeneity to a total mass percentage concentration of the wall material of 1.5%-2.5% in water, so as to obtain the wall material solution.

Preferably, the specific operation of the step IV is as follows:
mixing the core material solution prepared in the step II with the wall material solution prepared in the step III, rapidly dispersing for 20-40 s with a high-speed homogenizer at 10000 r/min, repeating for 3 times, and then stirring for 5-10 min with a magnetic stirrer at a speed of 800-1000 r/min; freezing the obtained mixed solution in a refrigerator at −80° C. for 12 to 24 hours, and freeze-drying the frozen solution in a vacuum freeze dryer for 12 to 24 hours; crushing the freeze-dried product, sieving the crushed product with a 100-mesh sieve, and collecting the undersize product to obtain the microcapsules, or crushing the freeze-dried product into powder by a superfine powder jet mill to obtain the microcapsules.

Preferably, the microcapsules have a particle size in a range from 850 to 1000 nm.

Preferably, the drug can also comprise pharmaceutically acceptable adjuvants.

The pharmaceutically acceptable adjuvants include, but not limited to: (1) diluent, such as starch, powdered sugar, dextrin, lactose, pregelatinized starch, microcrystalline fiber, inorganic calcium salt (such as calcium sulfate, calcium hydrogen phosphate, pharmaceutical calcium carbonate, etc.), mannitol, vegetable oil, polyethylene glycol, distilled water, etc.; (2) binder, such as distilled water, ethanol, starch slurry, sodium carboxymethyl cellulose, hydroxypropyl cellulose, methyl cellulose and ethyl cellulose, hydroxypropyl methylcellulose, etc; (3) disintegrant, such as dry starch, sodium carboxymethyl starch, low-substituted hydroxypropyl cellulose, crospolyvinylpyrrolidone, croscarmellose sodium, etc; (4) lubricant, such as magnesium stearate, aerosil, talc, hydrogenated vegetable oil, polyethylene glycols, magnesium lauryl sulfate, etc.

Preferably, the drug is a clinically acceptable formulation.

More preferably, the drug is an oral formulation.

Preferably, the oral formulation is selected from one of the following: a tablet, a capsule, a granule, a dry suspension, a suspension and an oral solution.

In the specification of the present application, “about” is intended to include values within the range of “the specified value ±25%”, unless otherwise specified. If the final concentration of nitrate is about 4 mg/ml in the step of preparing the core material solution, the final concentration of nitrate in the core material solution may be 4±1 mg/ml, for example, any concentration in the range from 3 mg/ml to 5 mg/ml.

The microcapsule prepared by the preparation method of the invention has high embedding rate. The highest embedding rate (based on nitrate) was 87.2%, as determined by HPLC.

Sustained release dynamics has demonstrated that controlled release can be realized by the microcapsule provided by the invention in simulated intestinal and gastric juice.

The NOD/Itj mouse is a spontaneous non-obese diabetic mouse, and the inventor found that the salivary gland of the NOD/Itj mouse had lymphatic infiltration at about 8 weeks, and the saliva decreased significantly at the 12th week. Therefore, the NOD/Itj mouse can be used as an animal model of SS. It has been demonstrated by a pharmacodynamic test that the microcapsules of the invention can obviously improve the salivary flow rate of the NOD/Itj mouse (P<0.01), and alleviate the infiltration of salivary glands by lymphocytes; and there is no obvious influence on the salivary flow rate of the NOD/Itj mouse by nitrate, vitamin C, and the mechanical mixture of nitrate and vitamin C administrated via gastric gavage. It is shown that the microcapsules of the present invention ensure the synergistic effect of vitamin C and nitrate in vivo, thereby treating SS.

### BRIEF DESCRIPTION OF DRA WINGS

The present invention will be further described with reference to the accompanying drawings.

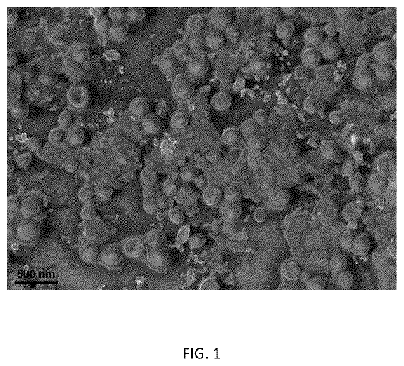

FIG. 1 shows a transmission electron microscopy photograph of the microcapsules prepared in Example 1 of the present invention.

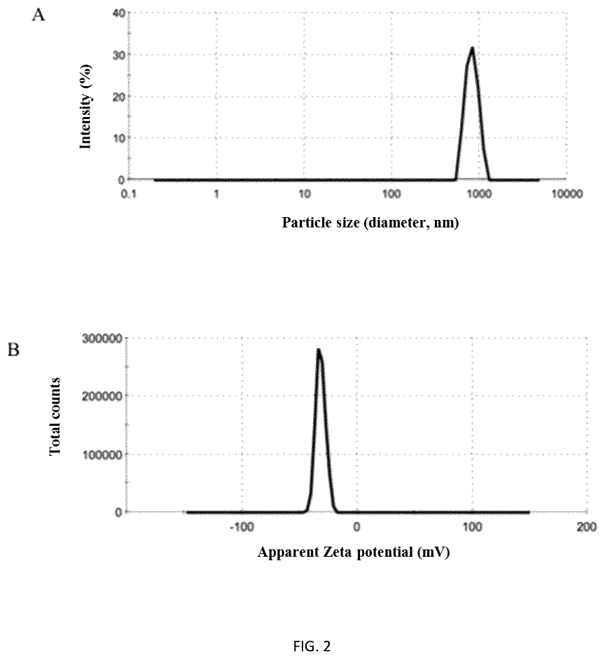

FIG. 2 shows the particle size distribution and the Zeta potential of the microcapsules prepared in Example 1 of the present invention, wherein FIG. 2A shows the particle size distribution and FIG. 2B shows the Zeta potential distribution.

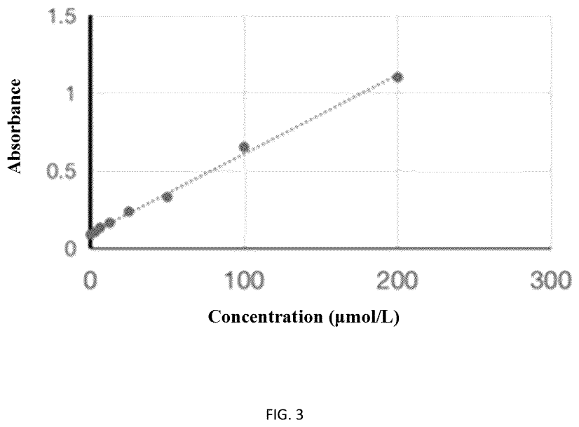

FIG. 3 shows a standard curve established for detection of the embedding rate of the microcapsules prepared in Example 1, wherein X axis represents the nitrate content and Y axis represents the absorbance value.

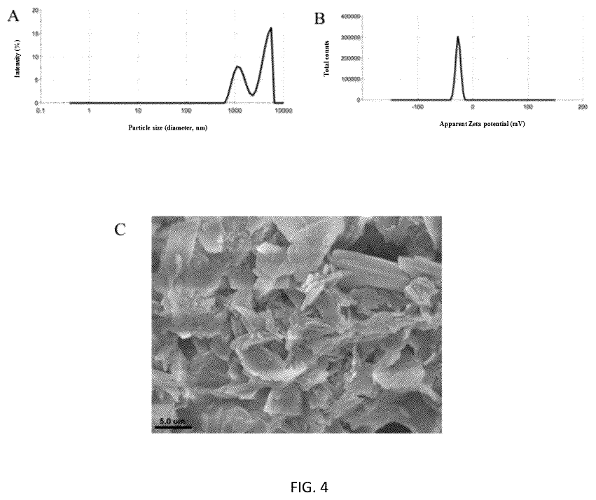

FIG. 4 shows the particle size distribution and the Zeta potential of the microcapsules prepared in Comparative Example 1, wherein FIGS. 4A, 4B and 4C shows the particle size distribution, the Zeta potential distribution, and the scanning electron microscopy photograph of the microcapsules, respectively.

FIG. 5 shows the cumulative release profile of the microcapsules prepared in Example 1 in simulated intestinal fluid and simulated gastric fluid as measured in Example 2, with time (h) on X axis and the cumulative release rate (%) on Y axis.

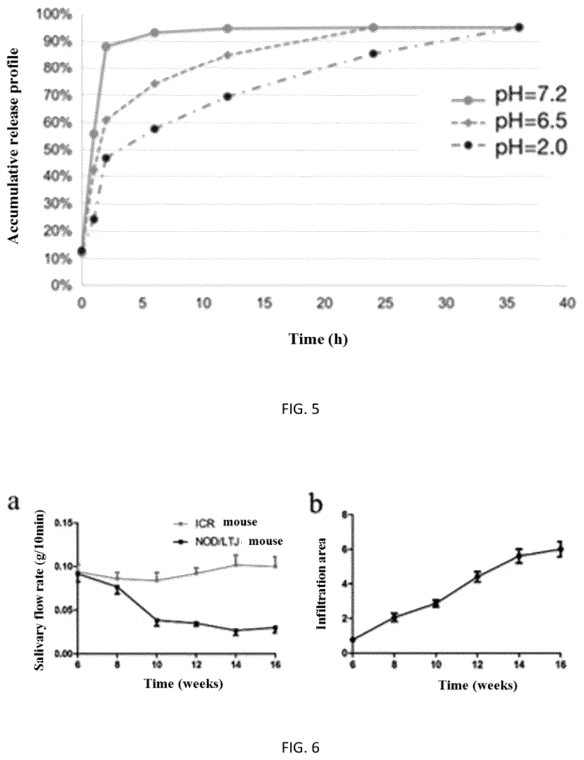

FIG. 6 shows the saliva flow rate and the lymphocyte infiltration area in submandibular glands (%) of the NOD/Itj mice measured in Example 5 from week 8 to week 14, wherein FIG. a on the left side shows that the saliva flow rate of the NOD/Itj mice gradually decreases, and FIG. b on the right side shows that the lymphocyte infiltration area in submandibular glands gradually increases.

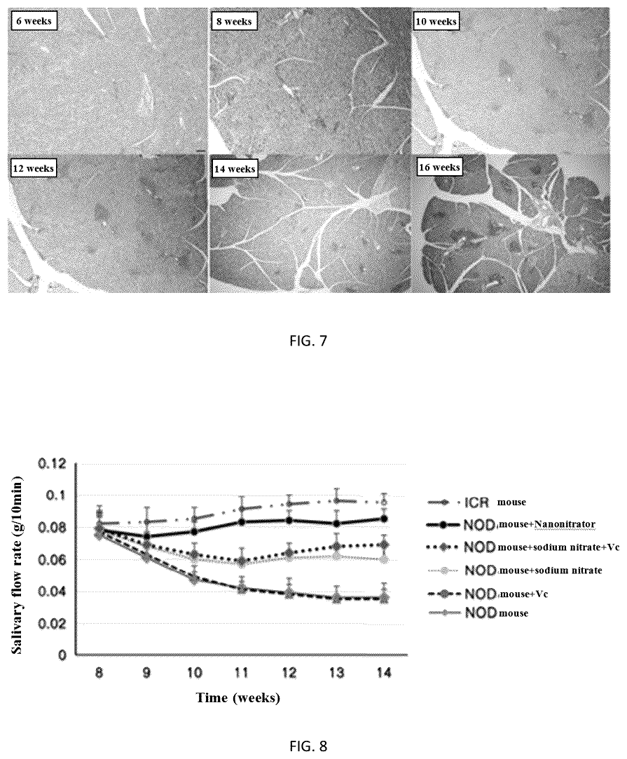

FIG. 7 shows microscopic photographs (×20) of the sections of the submandibular glands of the NOD/Itj mice stained with HE at 6, 8, 10, 12, 14, and 16 weeks in Example 5. The figure shows a gradual increase in the number of lymphocyte infiltration foci in the submandibular glands of the mouse.

FIG. 8 shows the saliva flow rates of the mice in each group measured in Example 5 from week 8 to week 14.

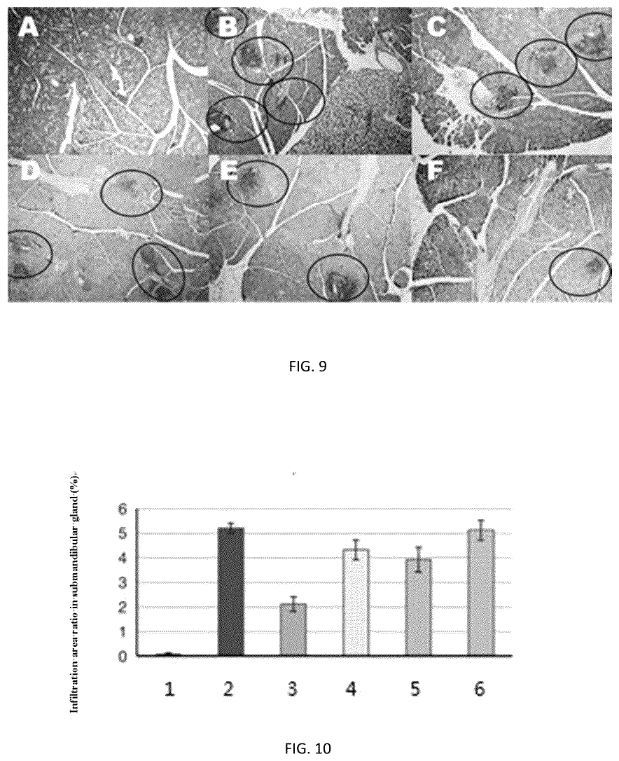

FIG. 9 is a photograph showing the sections of the submandibular glands of the mice in each group at week 14, in which the circled area is a lymphocyte infiltration area.

FIG. 10 is a histogram showing the relative lymphocyte infiltration area (%) of the submandibular glands of the mice in each group at week 14, in which:
1: ICR mouse,
2: NOD/Itj mouse,
3: NOD/Itj mouse+microcapsules,
4: NOD/ltj mouse+sodium nitrate,
5: NOD/Itj mouse+sodium nitrate+Vc,
6: NOD/Itj mouse+Vc.

### DETAILED DESCRIPTION

The present invention will be described below with reference to specific Examples. It will be understood by those skilled in the art that these embodiments are merely illustrative of the invention and do not limit the scope of the invention in any way.

The experimental methods in the following Examples are all conventional methods, unless otherwise specified. The raw materials, reagents, and the like used in the following Examples are commercially available, unless otherwise specified.

### Example 1 Preparation of a Microcapsule of Nitrate and Vitamin C

### 1.1 Reagents and Instruments

Pectin, sodium carboxymethyl cellulose (CMC-Na), Nantong Tailida New Materials Co., Ltd.; chitosan (chitosan 3000, and chitosan 75000), Anhui Yuanzheng Bioengineering Co., Ltd.; vitamin C, sodium nitrate, Merck Ltd. (Sigma-Aldrich); sodium heptanesulfonate, vitamin standard, Shanghai Yuanye Bio-Technology Co., Ltd; EDTA, isopropanol, triethylamine, glacial acetic acid, ethanol, Beijing Reagent Company.

Electronic balance BSA124, Sartorius Scientific Instruments Co., Ltd.; rotary evaporator LABOROTA4003, Heidolph Instruments GmbH & Co. KG (Germany); 101-0 electro-thermostatic blast oven, Tianjin Taisite Instrument Co., Ltd.; magnetic stirrer, ultrafine pulverizer, IKA Instrument Co., Ltd. (Germany); ultrafine jet mill, Model: R & D Jet Mills Lab; KQ-500DE digital control ultrasonic cleaner, Kunshan Ultrasonic Instruments Co., Ltd.; Vacuum freeze dryer Alpha1-4LDplus, MARTIN CHRIST corporation (Germany); Agilent 1200 HPLC, Agilent Technologies (U.S.).

### 1.2 Preparation of Microcapsules

### I. Preparation of the Raw Materials

14 mg of vitamin C, 27.5 mg of sodium nitrate, 95 mg of CMC-Na, 95 mg of pectin and 112 mg of chitosan (chitosan 3000) were weighed out.

### II. Preparation of the Core Material Solution

Vitamin C were placed in a flask, in to which 5 ml of pure water was added. The vitamin C was thoroughly dissolved in a water bath at 20-25° C. under dark, and then cooled to 4° C. in a refrigerator. Subsequently, sodium nitrate and chitosan were added and dissolved by stirring, so as to obtain a solution with a final nitrate concentration of 4 mg/mL, i.e., the core material solution.

### III. Preparation of the Wall Material Solution

CMC-Na was added into 5 ml pure water, heated in a water bath at 80° C. and stirred with a magnetic stirrer until it was completely dissolved to obtain solution A. Pectin was added into 4.7 ml of pure water, heated in a water bath at 50° C., and stirred until the pectin was completely dissolved to obtain solution B. The solution A and the solution B were mixed and stirred homogeneously to obtain the wall material solution, in which the total mass percentage concentration of the wall material in the water is 1.95%.

### IV. Preparation of the Microcapsules

The wall material solution and the core material solution were mixed and quickly dispersed by a high-speed homogenizer at a speed of 10000 r/min 30 s. The process was repeated for 3 times. The solution was then stirred by a magnetic stirrer to make it thoroughly mixed. The mixture was poured into a culture dish to a thickness of 2-5 cm, which was then placed in a refrigerator at −80° C. and frozen for 12-24 hours. The frozen solution was then freeze-dried in a vacuum freeze drier (Alpha1-4LDplus, MARTIN CHRIST corporation, Germany) for 12-24 hours. After freeze drying, the sample existed in flocculent structure. The flocculent sample was then pulverized into powder by a superfine jet mill to obtain the microcapsules. The resulting microcapsules were placed in a desiccator for later use.

### 1.3 Characterization and Related Tests of Microcapsules

### A. Morphological Observation

This product is light yellow powder. It was observed by a scanning electron microscope that the particle size was-890 nm with a relatively regular spheroid. The transmission electron microscopy photograph was shown in FIG. 1.

### B. Determination of Particle Size and Zeta Potential

The particle size of the microcapsules prepared in Example 1 was measured using a laser nanoparticle size analyzer, and the Zeta potential of the microcapsules prepared in Example 1 was measured using a Zeta potential analyzer. The results were each shown in FIG. 2. As shown in FIG. 2A, the particle size of the microcapsules is normally distributed with the average particle size of 890.2±180.4 nm; while in FIG. 2B, the Zeta potential of the microcapsules was −31.5±4.2 mV. The results showed that the microcapsules prepared in Example 1 was uniform nanoscale particles and suitable for sustained release.

### C. Determination of Embedding Rate

Since NO₃⁻ is the main active component, it was taken as the main analysis object. The overall embedding effect of the microcapsules is reflected by the content and embedding rate of nitrate in the microcapsules.

C-1. Determination of the content of nitrate not embedded in the surface layer of the microcapsules:

30 mg of the microcapsules were placed into a beaker under dark, and washed by 15 mL 50% ethanol aqueous solution for 3 times. The supernatant was filtered, and the filtrates were combined. The combined filtrate was then centrifuged for 5 min at 3500 r/min and at 4° C. The supernatant after centrifugation was rotary evaporated under vacuum to dryness. Finally, the dried material was redissolved with 10 mL of pure water, and passed through a 0.45 μm organic filter membrane. The filtrate was then placed in a brown vial and stored at 4° C. for later use.

The nitrate content was determined using a total nitric oxide and nitrate/nitrite assay kit (PKGE001, R & D Systems, USA):

### Preparation:

1) The sample was placed at 4° C. for 2 h and centrifuged at 14000 rpm for 10 min, and the supernatant was pipetted out.
2) The resulting supernatant was filtered and 10-fold diluted to obtain reaction solution 1.
3) Preparation of reaction solution 2: the reaction solution 1 was 10-fold diluted using distilled water/deionized water to obtain reaction solution 2.
4) Preparation of nitrate reductase: the nitrate reductase solution was prepared with 1.0 mL of nitrate reductase stock solution, was vortexed strongly, stood at room temperature for 15 min, was vortexed and then stood at room temperature for another 15 min, was vortexed again and used immediately.

The nitrate reductase solution was diluted with the reaction solution 2 to prepare a nitrate reductase solution with the concentration of ⅕ of the stock solution. The steps were as follows:
a. Nitrate reductase (×well+2)×5 μL;
b. The reaction solution 2 was taken out with a volume equal to 4 folds of that of the solution used in the step a;
c. The solutions of steps a and b were added to a clean test tube, and vortexed;
d. It was placed on ice and used within 15 min.
5) NADH reagent—NADH was prepared with 5.0 mL of deionized or distilled water. The solution was allowed to stand for 3 minutes with gentle stirring before use. The solution should be used within 15 minutes or placed on ice.
6) Preparation of nitrate standard:
900 μL of the reaction solution 2 was pipetted into a 200 μmol/L tube, and a series of nitrate standards at the following concentrations were prepared: 100 μmol/L, 50 μmol/L, 25 μmol/L, 12.5 μmol/L, 6.25 μmol/L, and 3.12 μmol/L. Reaction solution 2 was used as blank (0 μmol/L).

Steps for detecting nitrate content (according to the instruction of the kit):
1) All reagents, standards, samples, and so on were prepared according to the previous preparation steps;
2) 100 μL of reaction solution 2 was added to the wells of the blank group;
3) 100 μL of the nitrate standard or the sample was each added to the remaining wells;
4) 50 μL NADH was added to all wells;
5) 50 μL of the diluted nitrate reductase was added to all wells, mixed well, and covered with tape;
6) Incubation at 37° C. for 30 min;
7) 100 μL of Griess I reaction solution was added to all wells;
8) 100 μL of Griess II reaction solution was added to all wells, sides of the plate were gently tapped, and the plate was mixed well;
9) Incubation at room temperature for 10 min;
10) Optical density (O.D.) was determined at a wavelength of 540 nm with a wavelength calibration of 690 nm;
11) A standard curve was established according to the measured values of the standards, and the contents of the nitrate in the samples in each group were calculated. The standard curve is shown in FIG. 3, and the regression equation is:

$$y = 0.0051 x + 0.0985 ( {R}^{2} = 0.9965 )$$

The result of the nitrate content on the surface of the microcapsules (not embedded) was shown in Table 1, where “n=6” indicated that 6 parallel tests were performed.

TABLE 1
Surface nitrate content of the microcapsules prepared in Example 1

| | Average value (μmol/L) | SD (μmol/L) |
|---|---|---|
| nitrate content | 280.1 | 21.7 |

Note:
n = 6

### C-2. Assay of the Total Nitrate Content in the Microcapsules:

30 mg of the microcapsules were placed into a beaker under dark, into which 10 mL of pure water was added. The microcapsules were dissolved with the aid of ultrasound for 3 times, and the suspension was centrifuged at 3500 r/min and at 4° C. for 5 min. A certain amount of the supernatant was pipetted from it. The same determination method as above was performed. The result was shown in Table 2, where “n=6” indicated that 6 parallel tests were performed.

TABLE 2
Total nitrate content in the microcapsules prepared in Example 1

| | Average value (μmol/L) | SD (μmol/L) |
|---|---|---|
| nitrate content | 2188.5 | 35.7 |

Note:
n = 6

### C-3. Calculation of Embedding Rate of the Microcapsules

The results obtained in C-1 and C-2 were substituted into the following equation:

$$Embedding Rate = ( 1 - the content of nitrate in the surface layer of microcapsules / the total content of nitrate in microcapsules ) \times 100 \%$$

After calculation, the embedding rate was 87.2% (n=6).

### Comparative Example 1. Preparation of Microcapsules of Nitrate and Vitamin C

The microcapsules of nitrate and vitamin C were prepared in this Comparative Example, in which the raw materials and methods were substantially the same as those in Example 1, except that the chitosan was chitosan 75000.

The particle size of the prepared microcapsules was measured by a laser nanoparticle analyzer, the Zeta potential of the microcapsules was measured by a Zeta potential analyzer, and the morphology of the microcapsules was observed by a scanning electron microscope. The results were shown in FIG. 4. In FIG. 4A, the particle size of the microcapsules prepared in this Comparative Example was not normally distributed, indicating that the size was not uniform. The particle size was close to 4408+943.1 nm. In FIG. 4B, the Zeta potential of the microcapsules prepared in this Comparative Example was reduced to −26.6 mV. In FIG. 4C, the microcapsules prepared using chitosan 75000 were irregular. As compared with the microcapsules of Example 1, the particle size of the microcapsules of this Comparative Example has increased significantly, reaching the micron level, suggesting that it may be trapped by liver and is not suitable for sustained release. The absolute value of Zeta potential of the microcapsules in Comparative Example 1 was less than 30 mV, suggesting that the microcapsules tended to aggregate after dispersion with poor system stability.

### Example 3. Pharmacokinetic Test of the Microcapsules of Example 1

The sustained release kinetics of the microcapsules prepared in Example 1 was studied in this Example.

### 1. In Vitro Test

Preparation of simulated gastric juice: 3.2 g of pepsin and 2 g of sodium chloride were added to 7 mL of hydrochloric acid, and water was then added to a total volume of 1000 mL. The pH value of the solution was 1.2.

Preparation of simulated intestinal fluid: 6.8 g of potassium dihydrogen phosphate was dissolved in 250 ml of water, into which 77 mL of 0.2 mL/L sodium hydroxide solution and 500 mL of water were added. Subsequently, 10 g of trypsin was added. After dissolution, the pH was adjusted to 6.8 with sodium hydroxide solution or 0.2 mol/L hydrochloric acid solution, and then it was diluted to 1000 mL with water.

Methods: 50 mg of the microcapsules were accurately weighed out and put into a reagent bottle, after which 100 mL of simulated intestinal juice or simulated gastric juice was added. The reagent bottle was placed on a magnetic stirrer and stirred at a speed of 100 r/min. 0.5 mL of the supernatant sample was collected every 3 minutes, and diluted 10 times, so as to determine the content of nitrate therein. After collection, the release rate of nitrate was determined by immediately supplementing 0.5 mL of simulated intestinal fluid or simulated gastric fluid, and the final release of nitrate from the microcapsules in simulated intestinal fluid and simulated gastric fluid was investigated.

The results were shown in FIG. 5. As shown in FIG. 5, the microcapsules were relatively stable in neutral solution, and in simulated intestinal fluid and simulated gastric fluid. The degree of sustained release of nitrate in the microcapsules was significantly enhanced, and was maintained stable.

### 2. In Vivo Test

Sodium nitrate, the microcapsules of Comparative Example 1, and the microcapsules of Example 1 were administered once at a dose of 2 mmol/kg sodium nitrate by gastric gavage. Blood was collected from vena caudalis at 0 h, and 2, 4, 6, 12, and 24 h after gastric gavage, respectively.

Nitrate detection method: the method was same as Example 1, except that the sample was the serum obtained from the tail vein blood of mouse after centrifugation. The results were shown in Table 3.

TABLE 3
Nitrate content in the blood of the mice
after single administration (μmol/L)

| | | Comparative | |
|---|---|---|---|
| Time (h) | NaNO₃ | example 1 | Example 1 |
| 0 | 41.8 | 42.2 | 42.7 |
| 2 | 1052.8 | 971.4 | 799.6 |
| 4 | 572.4 | 702.8 | 833.9 |
| 6 | 297.4 | 475.0 | 883.1 |
| 12 | 45.3 | 165.9 | 402.4 |
| 24 | 32.7 | 30.9 | 95.26 |

The data in Table 3 showed that the microcapsules of the invention could achieve a sustained release and maintain an effective plasma concentration of nitrate for 6 to 12 hours as compared to the microcapsules prepared in Comparative Examples 1.

### Example 4. Prophylactic and Therapeutic Effects of the Microcapsules of the Invention on SS

The NOD/Itj mouse is a spontaneous non-obese diabetic mouse, and the inventor found that the salivary gland of the NOD/Itj mouse had lymphatic infiltration at about 8 weeks, and the saliva decreased significantly at the 12th week. Therefore, the NOD/Itj mouse can be used as an animal model of SS. Subsequently, the microcapsules of the present invention were administered by gastric gavage, the saliva flow rate was monitored, and the experimental samples were collected at the 12th week to observe the prophylactic and therapeutic effects of the microcapsules of the present invention on SS.

### 1. Changes of Salivary Secretion in NOD/Itj Mice

The salivary flow rate was measured every two weeks from the 8th week after birth, and the saliva was collected. At the same time, the submandibular gland samples were collected and stained with HE for histological observation, and the degree of lymphocyte infiltration was analyzed by Image J.

Method for Collecting Whole Saliva from the Mouse:

After proper anaesthesia, the mice were injected I.P. with pilocarpine nitrate in aseptic normal saline at a concentration of 0.4 mg/ml and a dosage of 0.5 mg/100 g b.w. After the injection, the mice were laid on a 20° inclined mouse board on prone position, with the head slightly inclined downward, so that the head was in a slightly lower position. About 5 minutes after the injection, after the first drop of saliva from the mouth of the mouse, after 5 minutes, the time was recorded and the saliva was collected, and the amount of saliva was collected for 20 minutes (the amount of saliva is detected by inserting a small sterile cotton ball with known weight under the tongue of mouse, and weighing cotton ball 20 minutes later).

### Methods of Collecting and Staining the Mouse Submandibular Gland:

After the experimental animals were sacrificed, they were placed on supine position, disinfected, and the neck was incised to dissect the submandibular gland completely. The tissues were washed with PBS, cut into 0.2 cmx 0.2 cm, and fixed in 4% paraformaldehyde (pH 7.2) at 4° C. for 24-48 h. After dehydration, wax impregnation and embedding, the tissues were sectioned at a thickness of 4 μm and baked, followed by hematoxylin and eosin (HE) staining. The remaining fresh samples were placed in a refrigerator at −80° C.

The results showed that the salivary flow rate of NOD/Itj mice decreased significantly from the eighth week to the lowest value at the 14th week, while the salivary flow rate of ICR mice in the control group remained stable (see the broken line graph on the left side of FIG. 6). The degree of lymphocytic infiltration in the submandibular gland of NOD/Itj mice gradually increased from the sixth week (see the broken line graph on the right side of FIG. 6, and the photographs of the stained sections of the submandibular gland tissue shown in FIG. 7).

The above results showed that NOD/Itj mice spontaneously developed lymphocyte infiltration in submandibular gland and a significant decrease in salivary flow rate. Thus, NOD/Itj mice can mimic (at least partially) SS and be used to study the therapeutic effect of the microcapsules of the present invention on SS.

### 2. Effect of the Microcapsules of the Invention on Salivary Flow Rate and Lymphocyte Infiltration in Submandibular Gland of NOD/Itj Mice

The microcapsules were prepared as described in Example 1, and their effects on salivary flow rate and lymphocyte infiltration in submandibular gland of NOD/Itj mice were investigated.

### 2.1 Test Animals

ICR mice: Beijing Vital River Laboratory Animal Technology Co., Ltd.

NOD/Itj mice: Beijing Vital River Laboratory Animal Technology Co., Ltd.

### 2.2 Groups and Administration

From the 8th week after birth, the mice in each group were administrated via gastric gavage, and the specific groups and dosages were shown in Table 4.

TABLE 4
Groups and administration of the microcapsules
of the invention for preventing and treating SS

| | Animals + | | | The number |
|---|---|---|---|---|
| Group | drugs | Dosage | | of animals |
| 1 | ICR mice + | Normal saline, 0.5 mL | | 10 |
| | normal saline | | | |
| 2 | NOD/ltj mice + | Normal saline, 0.5 mL | | 10 |
| | normal saline | | | |
| 3 | NOD/ltj mice + | 2 | mmol/kg | 10 |
| | Vc | | | |
| 4 | NOD/ltj mice + | 0.4 | mmol/kg | 10 |
| | NaNO₃ | | | |

| | | | |
|---|---|---|---|
| 5 | NOD/ltj mice + | NaNO₃ 0.4 mmol/kg + | 10 |
| | NaNO₃ + Vc | Vc 2 mmol/kg | |
| 6 | NOD/ltj mice + | 0.4 mmol/kg, based | 10 |
| | microcapsule | on NO₃_{^{−}} | |
| | of example 1 | | |

### 2.3 Test Method

The mice in each group were administrated with the drug by gastric gavage from the 8th. week after birth, and ICR mice and NOD/Itj mice were used as controls. Saliva flow rate was measured weekly. At the 14th week, the submandibular glands were collected, stained and observed histologically to analyze the degree of lymphocyte infiltration.

### 2.4 Results

The saliva flow rates of the mice in each test group at weeks 8-14 were shown in Table 5 and FIG. 8.

TABLE 5
Salivary flow rates of the mice in each group (g/10 min, mean value ± SD)

| | | | NOD/Itj | NOD/Itj | NOD/Itj | NOD/Itj |
|---|---|---|---|---|---|---|
| | ICR | NOD/Itj | mice + | mice + | mice + | mice + sodium |
| Weeks | mice | mice | microcapsules | sodium nitrate | Vc | nitrate + Vc |
| 8 | 0.082 ± 0.011 | 0.075 ± 0.012 | 0.078 ± 0.010 | 0.078 ± 0.011 | 0.077 ± 0.012 | 0.079 ± 0.012 |
| 9 | 0.083 ± 0.009 | 0.061 ± 0.006 | 0.074 ± 0.009 | 0.068 ± 0.006 | 0.063 ± 0.008 | 0.069 ± 0.011 |
| 10 | 0.085 ± 0.007 | 0.047 ± 0.005 | 0.077 ± 0.006 | 0.060 ± 0.004 | 0.049 ± 0.007 | 0.063 ± 0.008 |
| 11 | 0.091 ± 0.008 | 0.042 ± 0.004 | 0.083 ± 0.008 | 0.057 ± 0.004 | 0.041 ± 0.008 | 0.059 ± 0.005 |
| 12 | 0.094 ± 0.005 | 0.039 ± 0.010 | 0.084 ± 0.006 | 0.061 ± 0.009 | 0.038 ± 0.006 | 0.064 ± 0.008^{b} |
| 13 | 0.096 ± 0.009 | 0.036 ± 0.007 | 0.081 ± 0.009^{a} | 0.062 ± 0.007 | 0.035 ± 0.008 | 0.068 ± 0.007^{b} |
| 14 | 0.095 ± 0.006 | 0.037 ± 0.009 | 0.085 ± 0.006^{a} | 0.059 ± 0.009 | 0.034 ± 0.007 | 0.069 ± 0.010^{b} |

Note:
^{a}Very significant difference as compared to NOD/Itj mice group, p < 0.01, and no significant difference as compared to ICR group;
^{b}significant difference as compared to NOD/Itj mice group, p < 0.01, but p < 0.05, as compared to ICR group.

Table 6, FIG. 9 and FIG. 10 showed the lymphocyte infiltration in submandibular gland of the mice in each group at the 14th week. FIG. 9 showed photographs of the submandibular gland sections in each group of mice at the 14th week, in which the circled area is the lymphocyte infiltration area. FIG. 10 was a histogram showing the relative lymphocyte infiltration area (%) in submandibular gland in each group of mice at week 14.

TABLE 6
Infiltration area (%) of the submandibular gland of the mice in each group at the 14^{th}

| | | | NOD/Itj | NOD/Itj | NOD/Itj | NOD/Itj |
|---|---|---|---|---|---|---|
| | ICR | NOD/Itj | mice + | mice + | mice + | mice + sodium |
| | mice | mice | microcapsules | sodium nitrate | Vc | nitrate + Vc |
| Infiltration | 0.31 ± 0.05 | 5.22 ± 0.21 | 2.19 ± 0.29^{a} | 4.42 ± 0.54^{b} | 5.16 ± 0.58 | 3.96 ± 0.48^{b} |
| area (%) | | | | | | |

Note:
^{a}Very significant difference as compared to NOD/Itj mice group, NOD/Itj mice + sodium nitrate group, NOD/Itj mice + sodium nitrate + Vc group and NOD/Itj mice + Vc group, p < 0.01;
^{b}significant difference as compared to NOD/Itj mice group, p < 0.05.

The concentration of nitrate ions in the plasma of mice in each group at week 14 was shown in Table 7.

TABLE 7
NO₃⁻ concentration (mmol/L) in the plasma of the mice in each group at the 14^{th} week

| | | | NOD/Itj | NOD/Itj | NOD/Itj | NOD/Itj |
|---|---|---|---|---|---|---|
| | ICR | NOD/Itj | mice + | mice + | mice + | mice + sodium |
| | mice | mice | microcapsules | sodium nitrate | Vc | nitrate + Vc |
| Nitrate ion | 38.5 ± 6.9 | 38.8 ± 6.8 | 97.9 ± 7.2^{a} | 74.6 ± 9.5^{b} | 75.2 ± 10.1^{b} | 39.9 ± 10.5 |
| concentration | | | | | | |

Note:
^{a}Very significant difference as compared to ICR mice group, NOD mice group, NOD/Itj mice + sodium nitrate group, NOD/Itj mice + sodium nitrate + Vc group and NOD/Itj mice + Vc group, p < 0.01;
^{b}significant difference as compared to ICR mice group and NOD mice group, p < 0.05.

### 2.5 Conclusion

The test results above showed that the microcapsules of nitrate and vitamin C of the invention could significantly increase the salivary flow rate of NOD/Itj mice and reduce the lymphocyte infiltration in submandibular gland, and the effect was significantly stronger than that of sodium nitrate, vitamin C and the combination of both sodium nitrate and vitamin C mixed directly. It has suggested that the microcapsules of nitrate and vitamin C provided by the invention can prevent and treat SS, especially xerostomia caused by SS.

1. Use of a microcapsule of nitrate and vitamin C in the preparation of a medicament for treating sicca syndrome, wherein the microcapsule comprises a wall material and a core material encapsulated in the wall material, wherein the core material comprises vitamin C and nitrate, and the molar ratio of nitrate ions to vitamin C is in the range from 1:1 to 5:1.

2. The use according to claim 1, wherein the use refers to the use of the microcapsule of the nitrate and vitamin C in the preparation of a medicament for treating xerostomia caused by sicca syndrome.

3. The use according to claim 1, wherein the molar ratio of nitrate ions to vitamin C is in the range from 1:1 to 4:1.

4. The use according to claim 3, wherein the molar ratio of nitrate ions to vitamin C is 4:1.

5. The use according to claim 4, wherein the nitrate is selected from the group consisting of sodium nitrate and/or potassium nitrate.

6. The use according to claim 5, wherein the nitrate is sodium nitrate.

7. The use according to claim 1, wherein the wall material comprises pectin and sodium carboxymethyl cellulose.

8. The use according to claim 1, wherein the core material further comprises chitosan.

9. The use according to claim 8, wherein the chitosan is chitosan 3000.

10. The use according to claim 1, wherein the raw materials of the microcapsule comprise the following components in parts by weight:
pectin 0.7-1.2 parts by weight, sodium carboxymethyl cellulose 0.7-1.2 parts by weight, chitosan 1 part by weight, and nitrate and vitamin C (in total) 0.3-1.2 parts by weight;
wherein the chitosan is chitosan 3000.

11. The use according to claim 10, wherein the mass ratio of pectin, sodium carboxymethyl cellulose and chitosan is 0.85:0.85:1.

12. The use according to claim 10, wherein the microcapsules are prepared by the following method:
I. Preparing the raw materials based on their proportions;
II. Preparing the core material solution by
dissolving vitamin C, nitrate and chitosan in water to a final concentration of nitrate of 4 mg/mL to obtain the core material solution;
III. Preparing the wall material solution by
uniformly mixing the wall material with water to a total mass percent concentration of the wall material in a range from 1.5 to 2.5% to obtain the wall material solution;
IV. Preparing the microcapsules by
uniformly mixing the core material solution prepared in step II and the wall material solution prepared in step III, freeze-drying, and crushing to obtain the microcapsules.

13. The use according to claim 12, the step II comprises:
dissolving said parts by weight of vitamin C in water in a 20-25° C. water bath in dark, cooling to 2-4° C., adding said parts by weight of nitrate and chitosan, stirring to dissolve them to a final concentration of nitrate of about 4 mg/mL to obtain the core material solution.

14. The use according to claim 12, the step III comprises:
dissolving said parts by weight of sodium carboxymethyl cellulose in water at 70-80° C., and stirring or homogenizing under high pressure to obtain solution A; dissolving said parts by weight of pectin in water at 45-55° C. to obtain solution B,
wherein the volume of the solution A is similar to that of the solution B; mixing the solution A and the solution B and stirring to homogeneity to a total mass percentage concentration of the wall material of 1.5%-2.5% in water, so as to obtain the wall material solution.

15. The use according to claim 12, wherein the step IV: comprises
mixing the core material solution prepared in the step II with the wall material solution prepared in the step III, rapidly dispersing for 20-40 s with a high-speed homogenizer at 10000 r/min, repeating for 3 times, and then stirring for 5-10 min with a magnetic stirrer at a speed of 800-1000 r/min; freezing the obtained mixed solution in a refrigerator at −80° C. for 12 to 24 hours, and freeze-drying the frozen solution in a vacuum freeze dryer for 12 to 24 hours; crushing the freeze-dried product, sieving the crushed product with a 100-mesh sieve, and collecting the undersize product to obtain the microcapsules, or crushing the freeze-dried product into powder by a superfine powder jet mill to obtain the microcapsules.

16. The use according to claim 1, wherein the microcapsules have a particle size of 850-1000 nm.

17. The use according to claim 1, wherein the drug is a clinically acceptable formulation.

18. The use according to claim 17, wherein the drug is an oral formulation.

19. The use according to claim 18, wherein the oral formulation is the one selected from the group consisting of a tablet, a capsule, a granule, a dry suspension, a suspension, and an oral liquid.
